SEQUENCE LISTING (7 sequences)

SEQ ID NO: 1 - DNA, 1499 bp, Bacillus subtilis
  CDS (1)..(891): alpha subunit
atgttaaccggattgaaaattgcagttatcggcggtgacgcaagacagctcgaaattataagaaagctcactgaacagcaggctgacatctatcttgtcggttttgaccaattggatcacggttttaccggggcagtaaaatgcaatattgatgaaattccttttcagcaaatagacagcatcattcttccagtatccgcgacaacaggagaaggtgtcgtatcgactgtattttcgaatgaagaagttgtgttaaaacaggaccatcttgacagaacgcctgcacattgtgtcattttctcaggaatttctaacgcctatttagaaaacattgcagctcaggcaaaaagaaaacttgttaagctgtttgagcgggatgacattgcgatatacaactctattccgacagtagaaggaacgatcatgctggctattcagcacacggattatacgatacacggatcacaggtggccgttctcggtctggggcgcaccgggatgacgattgcccgtacatttgccgcgctcggggcgaatgtaaaagtgggggcaagaagttcagcgcatctggcacgtatcactgaaatggggctcgttccttttcataccgatgagctgaaagagcatgtaaaagatatagatatttgcattaataccataccgagtatgattttaaatcaaacggtactttctagcatgacaccaaaaaccttaatattggatctggcctcacgtcccgggggaacggattttaaatatgccgagaaacaagggattaaagcacttcttgctcccgggcttccagggattgtcgctcctaaaacagctgggcaaatccttgcaaacgtcttgagcaagcttttggctgaaatacaagctgaggaggggaaataaggatgtcgtcattaaaaggaaaaagaatcgggtttgggctgaccgggtcgcattgcacatatgaagcggttttcccgcaaattgaggagttggtcaacgaaggagctgaagtccgtccggttgtcacatttaatgtaaaatctacaaatacccgatttggagagggcgcagaatgggttaaaaaaattgaagacctgactggatatgaggccattgattcgattgtaaaggcagaacctcttgggccgaagctgccccttgactgcatggtcattgcgcctttaacaggcaattcaatgagcaagctggcaaatgccatgacggacagcccggtgctgatggcggcaaaagcgacaatccggaacaatcggcctgtcgttctgggtatctcgacaaatgatgctcttggtttaaacggaacaaatttaatgaggctcatgtcaacaaaaaatatcttttttattccattcgggcaagatgatccatttaaaaaaccgaattcaatggtagccaaaatggatctgcttccgcaaacgattgaaaaggcactcatgcaccagcagcttcagccgattctagttgagaattatcagggaaatgactaa

SEQ ID NO: 2 - protein, 297 aa, Bacillus subtilis
MLTGLKIAVIGGDARQLEIIRKLTEQQADIYLVGFDQLDHGFTGAVKCNIDEIPFQQIDSIILPVSATTGEGVVSTVFSNEEVVLKQDHLDRTPAHCVIFSGISNAYLENIAAQAKRKLVKLFERDDIAIYNSIPTVEGTIMLAIQHTDYTIHGSQVAVLGLGRTGMTIARTFAALGANVKVGARSSAHLARITEMGLVPFHTDELKEHVKDIDICINTIPSMILNQTVLSSMTPKTLILDLASRPGGTDFKYAEKQGIKALLAPGLPGIVAPKTAGQILANVLSKLLAEIQAEEGK

SEQ ID NO: 3 - protein, 200 aa, Bacillus subtilis
MSSLKGKRIGFGLTGSHCTYEAVFPQIEELVNEGAEVRPVVTFNVKSTNTRFGEGAEWVKKIEDLTGYEAIDSIVKAEPLGPKLPLDCMVIAPLTGNSMSKLANAMTDSPVLMAAKATIRNNRPVVLGISTNDALGLNGTNLMRLMSTKNIFFIPFGQDDPFKKPNSMVAKMDLLPQTIEKALMHQQLQPILVENYQGND

SEQ ID NO: 4 - DNA, 1752 bp, Artificial
  Synthetic spoVF gene
tagctgccaattattccgggcttgtgacccgctacccgataaataggtcggctgaaaaatttcgttgcaatatcaacaaaaaggcctatcattgggaggtgtcgcaccaagtacttttgcgaagcgccatctgacggattttcaaaagatgtatatgctcggtgcggaaacctacgaaaggattttttacccatgctgaccggcctgaagatcgcagtgatcggcggcgatgcacgccagctggaaatcatccgcaagctgaccgaacagcaggcagatatctacctggtgggcttcgatcagctggatcacggcttcaccggcgcagtgaagtgcaacatcgatgaaatcccattccagcagatcgattccatcatcctgccagtgtccgcaaccaccggcgaaggcgtggtgtccaccgtgttctccaacgaagaagtggtgctgaagcaggatcacctggatcgcaccccagcacactgcgtgatcttctccggcatctccaacgcatacctggaaaacatcgcagcacaggcaaagcgcaagctggtgaagctgttcgaacgcgatgatatcgcaatctacaactccatcccaaccgtggaaggcaccatcatgctggcaatccagcacaccgattacaccatccacggctcccaggtggcagtgctgggcctgggccgcaccggcatgaccatcgcacgcaccttcgcagcactgggcgcaaacgtgaaggtgggcgcacgctcctccgcacacctggcacgcatcaccgaaatgggcctggtgccattccacaccgatgaactgaaggaacacgtgaaggatatcgatatctgcatcaacaccatcccatccatgatcctgaaccagaccgtgctgtcctccatgaccccaaagaccctgatcctggatctggcatcccgcccaggcggcaccgatttcaagtacgcagaaaagcagggcatcaaggcactgctggcaccaggcctgccaggcatcgtggcaccaaagaccgcaggccagatcctggcaaacgtgctgtccaagctgctggcagaaatccaggcagaagaaggcaagtaaggatgtcctccctgaagggcaagcgcatcggcttcggcctgaccggctcccactgcacctacgaagcagtgttcccacagatcgaagaactggtgaacgaaggcgcagaagtgcgcccagtggtgaccttcaacgtgaagtccaccaacacccgcttcggcgaaggcgcagaatgggtgaagaagatcgaagatctgaccggctacgaagcaatcgattccatcgtgaaggcagaaccactgggcccaaagctgccactggattgcatggtgatcgcaccactgaccggcaactccatgtccaagctggcaaacgcaatgaccgattccccagtgctgatggcagcaaaggcaaccatccgcaacaaccgcccagtggtgctgggcatctccaccaacgatgcactgggcctgaacggcaccaacctgatgcgcctgatgtccaccaagaacatcttcttcatcccattcggccaggatgatccattcaagaagccaaactccatggtggcaaagatggatctgctgccacagaccatcgaaaaggcactgatgcaccagcagctgcagccaatcctggtggaaaactaccagggcaacgattaaagttctgtgaaaaacaccgtggggcagtttctgcttcgcggtgttttttatttgtggggca

SEQ ID NO: 5 - protein, 297 aa, Artificial; identical to SEQ ID NO: 2
  Synthetic Construct

SEQ ID NO: 6 - protein, 200 aa, Artificial; identical to SEQ ID NO: 3
  Synthetic Construct

SEQ ID NO: 7 - DNA, 5091 bp, Artificial
  Plasmide
tcgatttaaatctcgagaggcctgacgtcgggcccggtaccacgcgtcatatgactagttcggacctagggatatcgtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgcgcaaagtcccgcttcgtgaaaattttcgtgccgcgtgattttccgccaaaaactttaacgaacgttcgttataatggtgtcatgaccttcacgacgaagtactaaaattggcccgaatcatcagctatggatctctctgatgtcgcgctggagtccgacgcgctcgatgctgccgtcgatttaaaaacggtgatcggatttttccgagctctcgatacgacggacgcgccagcatcacgagactgggccagtgccgcgagcgacctagaaactctcgtggcggatcttgaggagctggctgacgagctgcgtgctcggccagcgccaggaggacgcacagtagtggaggatgcaatcagttgcgcctactgcggtggcctgattcctccccggcctgacccgcgaggacggcgcgcaaaatattgctcagatgcgtgtcgtgccgcagccagccgcgagcgcgccaacaaacgccacgccgaggagctggaggcggctaggtcgcaaatggcgctggaagtgcgtcccccgagcgaaattttggccatggtcgtcacagagctggaagcggcagcgagaattatcgcgatcgtggcggtgcccgcaggcatgacaaacatcgtaaatgccgcgtttcgtgtgccgtggccgcccaggacgtgtcagcgccgccaccacctgcaccgaatcggcagcagcgtcgcgcgtcgaaaaagcgcacaggcggcaagaagcgataagctgcacgaatacctgaaaaatgttgaacgccccgtgagcggtaactcacagggcgtcggctaacccccagtccaaacctgggagaaagcgctcaaaaatgactctagcggattcacgagacattgacacaccggcctggaaattttccgctgatctgttcgacacccatcccgagctcgcgctgcgatcacgtggctggacgagcgaagaccgccgcgaattcctcgctcacctgggcagagaaaatttccagggcagcaagacccgcgacttcgccagcgcttggatcaaagacccggacacggagaaacacagccgaagttataccgagttggttcaaaatcgcttgcccggtgccagtatgttgctctgacgcacgcgcagcacgcagccgtgcttgtcctggacattgatgtgccgagccaccaggccggcgggaaaatcgagcacgtaaaccccgaggtctacgcgattttggagcgctgggcacgcctggaaaaagcgccagcttggatcggcgtgaatccactgagcgggaaatgccagctcatctggctcattgatccggtgtatgccgcagcaggcatgagcagcccgaatatgcgcctgctggctgcaacgaccgaggaaatgacccgcgttttcggcgctgaccaggctttttcacataggctgagccgtggccactgcactctccgacgatcccagccgtaccgctggcatgcccagcacaatcgcgtggatcgcctagctgatcttatggaggttgctcgcatgatctcaggcacagaaaaacctaaaaaacgctatgagcaggagttttctagcggacgggcacgtatcgaagcggcaagaaaagccactgcggaagcaaaagcacttgccacgcttgaagcaagcctgccgagcgccgctgaagcgtctggagagctgatcgacggcgtccgtgtcctctggactgctccagggcgtgccgcccgtgatgagacggcttttcgccacgctttgactgtgggataccagttaaaagcggctggtgagcgcctaaaagacaccaagggtcatcgagcctacgagcgtgcctacaccgtcgctcaggcggtcggaggaggccgtgagcctgatctgccgccggactgtgaccgccagacggattggccgcgacgtgtgcgcggctacgtcgctaaaggccagccagtcgtccctgctcgtcagacagagacgcagagccagccgaggcgaaaagctctggccactatgggaagacgtggcggtaaaaaggccgcagaacgctggaaagacccaaacagtgagtacgcccgagcacagcgagaaaaactagctaagtccagtcaacgacaagctaggaaagctaaaggaaatcgcttgaccattgcaggttggtttatgactgttgagggagagactggctcgtggccgacaatcaatgaagctatgtctgaatttagcgtgtcacgtcagaccgtgaatagagcacttaaggtctgcgggcattgaacttccacgaggacgccgaaagcttcccagtaaatgtgccatctcgtaggcagaaaacggttcccccgtagggtctctctcttggcctcctttctaggtcgggctgattgctcttgaagctctctaggggggctcacaccataggcagataacgttccccaccggctcgcctcgtaagcgcacaaggactgctcccaaagatcttcaaagccactgccgcgactgccttcgcgaagccttgccccgcggaaatttcctccaccgagttcgtgcacacccctatgccaagcttctttcaccctaaattcgagagattggattcttaccgtggaaattcttcgcaaaaatcgtcccctgatcgcccttgcgacgttggcgtcggtgccgctggttgcgcttggcttgaccgacttgatcagcggccgc

(19) United States
(12) Patent Application Publication
(10) Pub. No.: US 2011/0003963 A1
(43) Pub. Date: Jan. 6, 2011

(54) METHOD FOR THE PRODUCTION OF DIPICOLINATE

(75) Inventors: Oskar Zelder, Speyer (DE); Weol Kyu Jeong, Gunsan (KR); Corinna Klopprogge, Mannheim (DE); Andrea Herold, Ketsch (DE); Hartwig Schröder, Nussloch (DE)

Correspondence Address: CONNOLLY BOVE LODGE & HUTZ, LLP, P O BOX 2207, WILMINGTON, DE, 19899, US

(73) Assignee: BASF SE

(21) Appl. No.: 12/865,895

(22) PCT Filed: Feb. 4, 2009
(86) PCT No.: PCT/EP09/00758

§ 371 Date: Aug. 3, 2010

(30) Foreign Application Priority Data
Feb. 4, 2008 (EP) 08151031.5

(51) Int. Cl. C08G 69/08; C12P 17/12; C07H 21/04; C12N 15/63; C12N 1/21

(52) U.S. Cl. 528/292; 435/122; 536/23.2; 435/320.1; 435/252.32

(57) ABSTRACT

The present invention relates to a novel method for the fermentative production of dipicolinate by cultivating a recombinant microorganism expressing an enzyme having dipicolinate synthetase activity. The present invention also relates to corresponding recombinant hosts, recombinant vectors, expression cassettes and nucleic acids suitable for preparing such hosts as well as a method of preparing polyester or polyamide copolymers making use of dipicolinate as obtained by fermentative production.

The present invention relates to a novel method for the fermentative production of dipicolinate by cultivating a recombinant microorganism expressing an enzyme having dipicolinate synthetase activity. The present invention also relates to corresponding recombinant hosts, recombinant vectors, expression cassettes and nucleic acids suitable for preparing such hosts as well as a method of preparing polyester or polyamide copolymers making use of dipicolinate as obtained by fermentative production.

### BACKGROUND OF THE INVENTION

Dipicolinic acid (CAS number 499-83-2), also known as pyridine-2,6-dicarboxylic acid or DPA, is used in different technical fields, for example as monomer in the synthesis of polyester or polyamide type of copolymers, precursor for pyridine synthesis, stabilizing agent for peroxides and peracids, for example t-butyl peroxide, dimethyl-cyclohexanon peroxide, peroxyacetic acid and peroxy-monosulphuric acid, ingredient for polishing solution of metal surfaces, stabilizing agent for organic materials susceptible to be deteriorated due to the presence of traces of metal ions (sequestrating effect), stabilizing agent for epoxy resins, and stabilizing agent for photographic solutions or emulsions (preventing the precipitation of calcium salts).

It is well known that DPA is biosynthesized in endospores of bacteria. An enzyme catalyzing the biosynthesis of DPA from dihydrodipicolinate is dipicolinate synthetase. Said enzyme has been isolated from Bacillus subtilis and further characterized. It is encoded by the spoVF operon (BG10781, BG10782)

The fermentative production of said commercially interesting chemical compound has not yet been described.

The object of the present invention is therefore to provide a suitable method for the fermentative production of dipicolinic acid or corresponding salts thereof.

### DESCRIPTION OF THE FIGURES

FIG. 1 depicts the plasmid map of the pClik5aMCS cloning vector.

FIG. 2 depicts the DNA sequence of the spoVF gene from B. subtilis with alpha-subunit underlined and beta-subunit double underlined.

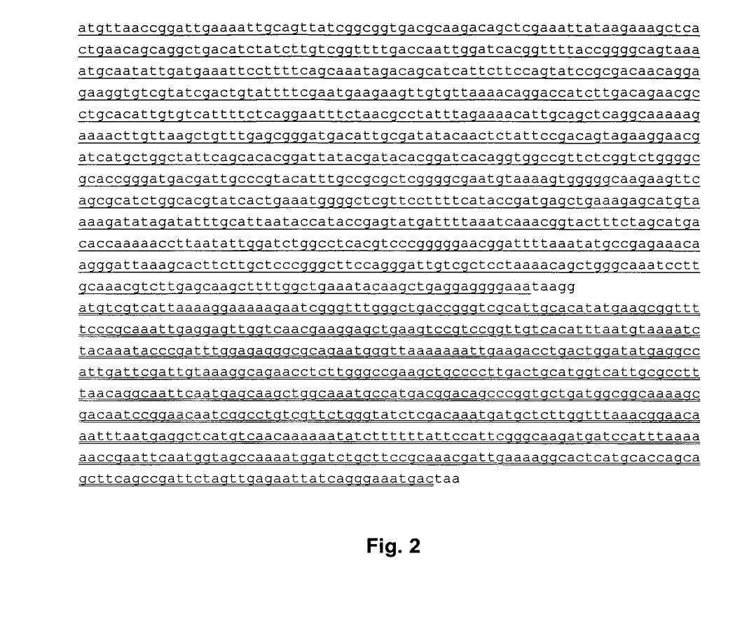

FIG. 3 depicts the DNA sequence of synthetic spoVF gene with N-terminal sod promoter in italics, with the alpha-subunit underlined and the beta-subunit double underlined, and with the groEL terminator in bold letters.

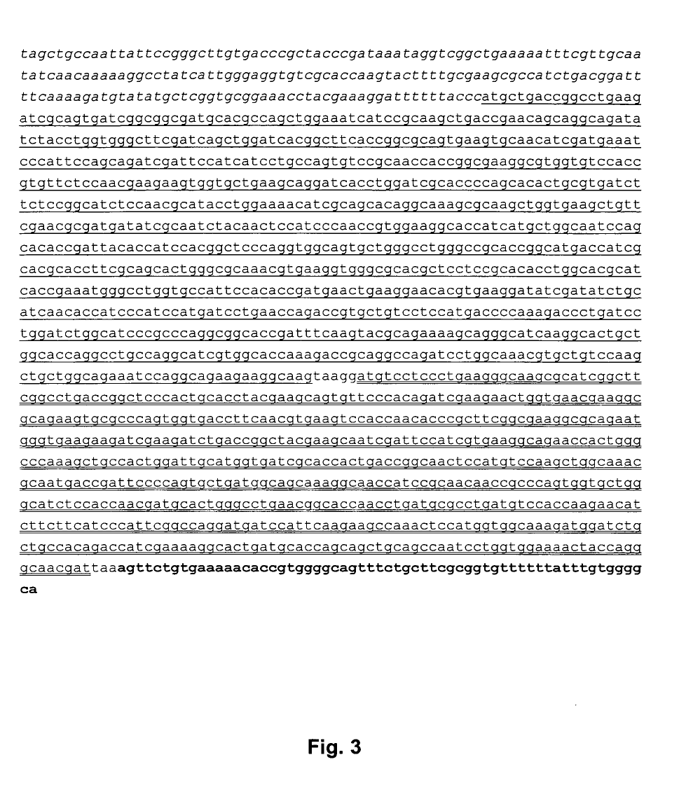

### SUMMARY OF THE INVENTION

The above-mentioned problem was solved by the present invention teaching the fermentative production of dipicolinate (dipicolinic acid or a salt thereof) by cultivating a recombinant microorganism expressing dipicolinate synthetase enzyme which enzyme converts dihydrodipicolinate that is formed in said microorganism as an intermediate during the course of the lysine biosynthetic pathway.

### DETAILED DESCRIPTION OF THE INVENTION

### 1. Preferred Embodiments

The present invention relates to a method for the fermentative production of DPA, which method comprises the cultivation of at least one recombinant microorganism which microorganism preferably being derived from a parent microorganism having the ability to produce lysine via the diaminopimelate (DAP) pathway with dihydrodipicolinate, in particular L-2,3-dihydrodipicolinate, as intermediary product, and which recombinant microorganism, qualitatively or quantitatively, retains said ability of said parent microorganism, and additionally having the ability to express heterologous dipicolinate synthetase, so that dihydrodipicolinate, in particular L-2,3-dihydrodipicolinate is converted into DPA. Said modified microorganism also may or may not retain its ability to produce lysine.

In particular, said parent microorganism is a lysine producing bacterium, preferably a coryneform bacterium. In particular, said parent microorganism is a bacterium of the genus Corynebacterium, as for example Corynebacterium glutamicum.

Said heterologous dipicolinate synthetase is of prokaryotic or eukaryotic origin. For example, said heterologous dipicolinate synthetase may originate from a bacterium of the genus Bacillus, in particular from Bacillus subtilis. Said Bacillus enzyme is composed of at least one alpha and at least one beta subunit.

The protein sequence of dipicolinate synthetase alpha chain is:

(SEQ ID NO: 2)
MLTGLKIAVIGGDARQLEIIRKLTEQQADIYLVGFDQLDHGFTGAVKC
NIDEIPFQQIDSIILPVSATTGEGVVSTVFSNEEVVLKQDHLDRTPAH
CVIFSGISNAYLENIAAQAKRKLVKLFERDDIAIYNSIPTVEGTIMLA
IQHTDYTIHGSQVAVLGLGRTGMTIARTFAALGANVKVGARSSAHLAR
ITEMGLVPFHTDELKEHVKDIDICINTIPSMILNQTVLSSMTPKTLIL
DLASRPGGTDFKYAEKQGIKALLAPGLPGIVAPKTAGQILANVLSKLL
AEIQAEEGK

The protein sequence of dipicolinate synthetase beta chain is:

(SEQ ID NO: 3)
MSSLKGKRIGFGLTGSHCTYEAVFPQIEELVNEGAEVRPVVTFNVKST
NTRFGEGAEWVKKIEDLTGYEAIDSIVKAEPLGPKLPLDCMVIAPLTG
NSMSKLANAMTDSPVLMAAKATIRNNRPVVLGISTNDALGLNGTNLMR
LMSTKNIFFIPFGQDDPFKKPNSMVAKMDLLPQTIEKALMHQQLQPIL
VENYQGND

The dipicolinate synthetase alpha-subunit has a calculated molecular weight of 31,947 Da and its beta subunit has a calculated molecular weight of 21,869 Da.

In a further embodiment of the method of the invention the heterologous dipicolinate synthetase comprises at least one alpha subunit having an amino acid sequence according to SEQ ID NO: 2 or a sequence having at least 80% identity thereto, as for example at least 85, 90, 92, 95, 96, 97, 98 or 99% sequence identity; and at least one beta subunit having an amino acid sequence according to SEQ ID NO: 3 or a sequence having at least 80% identity thereto, as for example at least 85, 90, 92, 95, 96, 97, 98 or 99% sequence identity.

The enzyme having dipicolinate synthetase activity may be encoded by a nucleic acid sequence, which is adapted to the codon usage of said parent microorganism having the ability to produce lysine.

For example, the enzyme having dipicolinate synthetase activity may be encoded by a nucleic acid sequence comprising
a) the spoVF gene sequence according to SEQ ID NO: 1, or
b) a synthetic spoVF gene sequence comprising a coding sequence essentially from residue 193 to residue 1691 according to SEQ ID NO: 4; or
c) any nucleotide sequence encoding a dipicolinate synthetase or its alpha and/or beta subunits as defined above.

In another embodiment of the method described herein at least one gene, as for example 1, 2, 3 or 4 genes, of the lysine biosynthesis pathway in said recombinant microorganism is deregulated in a suitable way, for example, in order to further support the formation of DPA.

Said at least one deregulated gene may be selected from aspartokinase, aspartatesemialdehyde dehydrogenase, dihydrodipicolinate synthase, dihydrodipicolinate reductase, pyruvate carboxylase, phosphoenolpyruvate carboxylase, glucose-6-phosphate dehydrogenase, transketolase, transaldolase, 6-phosphogluconolactonase, fructose 1,6-biphosphatase, homoserine dehydrogenase, phophoenolpyruvate carboxykinase, succinyl-CoA synthetase, methylmalonyl-CoA mutase, tetrahydrodipicolinate succinylase, succinyl-amino-ketopimelate transaminase, succinyl-diamino-pimelate desuccinylase, diaminopimelate epimerase, diaminopimelate dehydrogenase, and diaminopimelate decarboxylase.

According to another embodiment, the dipicolinate thus produced is isolated from the fermentation broth by well-known methods.

The present invention also relates to
nucleic acid sequences comprising the coding sequence for a dipicolinate synthetase as defined above;
expression cassettes, comprising at least one nucleic acid sequence as defined above which sequence is operatively linked to at least one regulatory nucleic acid sequence;
recombinant vectors, comprising at least one expression cassette as defined above; and
prokaryotic or eukaryotic hosts, transformed with at least one vector as defined above.

Preferably said host may be selected from recombinant coryneform bacteria, especially a recombinant Corynebacterium, in particular recombinant Corynebacterium glutamicum.

According to another embodiment, the present invention relates to a method of preparing a polymer, as for example a polyester or polyamide copolymer, which method comprises
a) preparing dipicolinate by a method as defined above;
b) isolating dipicolinate; and
c) polymerizing said dipicolinate with at least one further polyvalent copolymerizable co-monomer, for example, selected from polyols and polyamines or mixtures thereof.

Finally, the present invention relates to the use of the dipicolinate as produced according to the present invention as monomer in the synthesis of polyester or polyamide type copolymers; precursor for pyridine synthesis; stabilizing agent for peroxides and peracids, as for example t-butyl peroxide, dimethyl-cyclohexanon peroxide, peroxyacetic acid and peroxy-monosulphuric acid; ingredient for polishing solution of metal surfaces; stabilizing agent for organic materials susceptible to be deteriorated due to the presence of traces of metal ions (sequestrating effect); stabilizing agent for epoxy resins; and stabilizing agent for photographic solutions or emulsions (in particular, by preventing the precipitation of calcium salts).

### 2. Explanation of Particular Terms

Unless otherwise stated the expressions “dipicolinate”, “dipicolinic acid”, “dipicolinic acid salt” and “DPA” are considered to be synonymous. The dipicolinate product as obtained according to the present invention may be in the form of the free acid, in the form of a partial or complete salt of said acid or in the form of mixtures of the acid and its salt.

A dipicolinic acid “salt” comprises for example metal salts, as for example zinc dipicolinate, mono- or di-alkalimetal salts of dipicolinic acid, like mono-sodium disodium, mono-potassium and di-potassium salts as well as alkaline earth metal salts as for example the calcium or magnesium salts.

The term “dihydrodipicolinate” comprises any stereo isomeric form thereof, either alone, i.e. in stereoisomerically pure form, or as combination stereoisomers. In particular said term means L-2,3-dihydrodipicolinate either alone, i.e. in stereoisomerically pure form, or as combination with another stereoisomer. The term “dihydrodipicolinate” also relates to the free acid, the partial or complete salt of said acid or to mixtures of the acid and its salt. “Salts” are as defined above for dipicolinic acid.

“Deregulation” has to be understood in its broadest sense, and comprises an increase or decrease of complete switch off of an enzyme (target enzyme) activity by different means well known to those in the art. Suitable methods comprise for example an increase or decrease of the copy number of gene and for enzyme molecules in an organism, or the modification of another feature of the enzyme affecting the its enzymatic activity, which then results in the desired effect on the metabolic pathway at issue, in particular the lysine biosynthetic pathway or any pathway or enzymatic reaction coupled thereto. Suitable genetic manipulation can also include, but is not limited to, altering or modifying regulatory sequences or sites associated with expression of a particular gene (e.g., by removing strong promoters, inducible promoters or multiple promoters), modifying the chromosomal location of a particular gene, altering nucleic acid sequences adjacent to a particular gene such as a ribosome binding site or transcription terminator, decreasing the copy number of a particular gene, modifying proteins (e.g., regulatory proteins, suppressors, enhancers, transcriptional activators and the like) involved in transcription of a particular gene and/or translation of a particular gene product, or any other conventional means of deregulating expression of a particular gene routine in the art (including but not limited to use of antisense nucleic acid molecules, or other methods to knock-out or block expression of the target protein).

The term “heterologous” or “exogenous” refers to proteins, nucleic acids and corresponding sequences as described herein, which are introduced into or produced (transcribed or translated) by a genetically manipulated microorganism as defined herein and which microorganism prior to said manipulation did not contain or did not produce said sequence. In particular said microorganism prior to said manipulation may not contain or express said heterologous enzyme activity, or may contain or express an endogenous enzyme of comparable activity or specificity, which is encoded by a different coding sequence or by an enzyme of different amino acid sequence, and said endogenous enzyme may convert the same substrate or substrates as said exogenous enzyme.

A “parent” microorganism of the present invention is any microorganism having the ability to produce lysine via a pathway, as in particular the diaminopimelate dehydrogenase (DAP) pathway, with a dihydrodipicolinate, in particular L-2,3-dihydrodipicolinate, as intermediary product.

A microorganism “derived from a parent microorganism” refers to a microorganism modified by any type of manipulation, selected from chemical, biochemical or microbial, in particular genetic engineering techniques. Said manipulation results in at least one change of a biological feature of said parent microorganism. As an example the coding sequence of a heterologous enzyme may be introduced into said organism. By said change at least one feature may be added to, replaced in or deleted from said parent microorganism. Said change may, for example, result in an altered metabolic feature of said microorganism, so that, for example, a substrate of an enzyme expressed by said microorganism (which substrate was not utilized at all or which was utilized with different efficiency by said parent microorganism) is metabolized in a characteristic way (for example, in different amount, proportion or with different efficiency if compared to the parent microorganism), and/or a metabolic final or intermediary product is formed by said modified microorganism in a characteristic way (for example, in different amount, proportion or with different efficiency if compared to the parent microorganism).

An “intermediary product” is understood as a product, which is transiently or continuously formed during a chemical or biochemical process, in a not necessarily analytically directly detectable concentration. Said “intermediary product” may be removed from said biochemical process by a second, chemical or biochemical reaction, in particular by a reaction catalyzed by a “dipicolinate synthetase” enzyme as defined herein.

The term “dipicolinate synthetase” refers to any enzyme of any origin having the ability to convert a metabolite of a lysine-producing pathway into dipicolinate. In particular said term refers to enzymes by which a dihydrodipicolinate compound, in particular L-2,3-dihydrodipicolinate, is converted into DPA.

A “recombinant host” may be any prokaryotic or eukaryotic cell, which contains either a cloning vector or expression vector. This term is also meant to include those prokaryotic or eukaryotic cells that have been genetically engineered to contain the cloned gene(s) in the chromosome or genome of the host cell. For examples of suitable hosts, see Sambrook et al., MOLECULAR CLONING: A LABORATORY MANUAL, Second Edition, Cold Spring Harbor Laboratory, Cold Spring Harbor, N.Y. (1989).

The term “recombinant microorganism” includes a microorganism (e.g., bacteria, yeast, fungus, etc.) or microbial strain, which has been genetically altered, modified or engineered (e.g., genetically engineered) such that it exhibits an altered, modified or different genotype and/or phenotype (e.g., when the genetic modification affects coding nucleic acid sequences of the microorganism) as compared to the naturally-occurring microorganism or “parent” microorganism which it was derived from.

As used herein, a “substantially pure” protein or enzyme means that the desired purified protein is essentially free from contaminating cellular components, as evidenced by a single band following polyacrylamide-sodium dodecyl sulfate gel electrophoresis (SDS-PAGE). The term “substantially pure” is further meant to describe a molecule, which is homogeneous by one or more purity or homogeneity characteristics used by those of skill in the art. For example, a substantially pure dipicolinate synthetase will show constant and reproducible characteristics within standard experimental deviations for parameters such as the following: molecular weight, chromatographic migration, amino acid composition, amino acid sequence, blocked or unblocked N-terminus, HPLC elution profile, biological activity, and other such parameters. The term, however, is not meant to exclude artificial or synthetic mixtures of dipicolinate synthetase with other compounds. In addition, the term is not meant to exclude dipicolinate synthetase fusion proteins optionally isolated from a recombinant host.

### 3. Other Embodiments of the Invention

### 3.1 Deregulation of Further Genes

The fermentative production of dipicolinate with a recombinant Corynebacterium glutamicum lysine producer expressing B. subtilis spoVF operon may be further improved if it is combined with the deregulation of at least one further gene as listed below.

| Enzyme (gene product) | Gene | Deregulation |
|---|---|---|
| Aspartokinase | ask | Releasing feedback inhibition by |
| | NCgl 0247 | point mutation (Eggeling et al., (eds.), |
| | | Handbook of Corynebacterium |
| | | glutamicum, pages 20.2.2 |
| | | (CRC press, 2005)) and amplification |
| Aspartatesemialdehyde dehydrogenase | asd | Amplification (EP1108790) |
| | NCgl 0248 | |
| Dihydrodipicolinate synthase | dapA | Amplification (EP0841395) |
| | NCgl 1896 | |
| Dihydrodipicolinate reductase | dapB | Attenuation, knock-out or silencing by |
| | NCgl 1898 | mutation or others |
| Pyruvate carboxylase | pycA | Releasing feedback inhibition by point |
| | NCgl 0659 | mutation (EP1108790) and |
| | | amplification |
| Phosphoenolpyruvate carboxylase | ppc | Amplification (EP358940) |
| | NCgl 1523 | |
| Glucose-6-phosphate dehydrogenase | zwf | Releasing feedback inhibition by point |
| | NCgl 1514 | mutation (US2003/0175911) |
| | | and amplification |
| Transketolase | tkt | Amplification (WO0104325) |
| | NCgl 1512 | |
| Transaldolase | tal | Amplification (WO0104325) |
| | NCgl 1513 | |
| 6-Phosphogluconolactonase | pgl | Amplification (WO0104325) |
| | NCgl 1516 | |
| Fructose 1,6-biphosphatase | fbp | Amplification (EP1108790) |
| | NCgl 0976 | |
| Homoserine dehydrogenase | hom | Attenuating by point mutation |
| | NCgl 1136 | (EP1108790) |
| Phophoenolpyruvate carboxykinase | pck | Knock-out or silencing by mutation or |
| | NCgl 2765 | others (US6872553) |
| Succinyl-CoA synthetase | sucC | Attenuating by point mutation |
| | NCgl 2477 | (WO05/58945) |
| Methylmalonyl-CoA mutase | NCgl 1472 | Attenuating by point mutation |
| | | (WO05/58945) |
| Tetrahydrodipicolinate succinylase | dapD | Attenuation |
| | NCgl 1061 | |
| Succinyl-amino-ketopimelate transaminase | dapC | Attenuation |
| | NCgl 1343 | |
| Succinyl-diamino-pimelate desuccinylase | dapE | Attenuation |
| | NCgl 1064 | |
| Diaminopimelate epimerase | dapF | Attenuation |
| | NCgl 1868 | |
| Diaminopimelate dehydrogenase | ddh | Attenuation |
| | NCgl 2528 | |
| Diaminopimelate decarboxylase | lysA | Attenuation |
| | NCgl 1133 | |

The genes and gene products as mentioned in said table are known in the art.

EP 1108790 discloses mutations in the genes of homoserinedehydrogenase and pyruvatecarboxylase, which have a beneficial effect on the productivity of recombinant corynebacteria in the production of lysine. WO 00/63388 discloses mutations in the gene of aspartokinase, which have a beneficial effect on the productivity of recombinant corynebacteria in the production of lysine. EP 1108790 and WO 00/63388 are incorporated by reference with respect to the mutations in these genes described above.

In the above table for every gene/gene product possible ways of deregulation of the respective gene are mentioned. The literature and documents cited in the row “Deregulation” of the table are herewith incorporated by reference with respect to gene deregulation. The ways mentioned in the table are preferred embodiments of a deregulation of the respective gene.

A preferred way of an “amplification” is an “up”—mutation which increases the gene activity e.g. by gene amplification using strong expression signals and/or point mutations which enhance the enzymatic activity.

A preferred way of an “attenuation” is a “down”—mutation which decreases the gene activity e.g. by gene deletion or disruption, using weak expression signals and/or point mutations which destroy or decrease the enzymatic activity.

### 3.2 Proteins According to the Invention

The present invention is not limited to the specifically mentioned proteins, but also extends to functional equivalents thereof.

“Functional equivalents” or “analogs” or “functional mutations” of the concretely disclosed enzymes are, within the scope of the present invention, various polypeptides thereof, which moreover possess the desired biological function or activity, e.g. enzyme activity.

For example, “functional equivalents” means enzymes, which, in a test used for enzymatic activity, display at least a 1 to 10%, or at least 20%, or at least 50%, or at least 75%, or at least 90% higher or lower activity of an enzyme, as defined herein.

“Functional equivalents”, according to the invention, also means in particular mutants, which, in at least one sequence position of the amino acid sequences stated above, have an amino acid that is different from that concretely stated, but nevertheless possess one of the aforementioned biological activities. “Functional equivalents” thus comprise the mutants obtainable by one or more amino acid additions, substitutions, deletions and/or inversions, where the stated changes can occur in any sequence position, provided they lead to a mutant with the profile of properties according to the invention. Functional equivalence is in particular also provided if the reactivity patterns coincide qualitatively between the mutant and the unchanged polypeptide, i.e. if for example the same substrates are converted at a different rate. Examples of suitable amino acid substitutions are shown in the following table:

| Original residue | Examples of substitution |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

“Functional equivalents” in the above sense are also “precursors” of the polypeptides described, as well as “functional derivatives” and “salts” of the polypeptides.

“Precursors” are in that case natural or synthetic precursors of the polypeptides with or without the desired biological activity.

The expression “salts” means salts of carboxyl groups as well as salts of acid addition of amino groups of the protein molecules according to the invention. Salts of carboxyl groups can be produced in a known way and comprise inorganic salts, for example sodium, calcium, ammonium, iron and zinc salts, and salts with organic bases, for example amines, such as triethanolamine, arginine, lysine, piperidine and the like. Salts of acid addition, for example salts with inorganic acids, such as hydrochloric acid or sulfuric acid and salts with organic acids, such as acetic acid and oxalic acid, are also covered by the invention.

“Functional derivatives” of polypeptides according to the invention can also be produced on functional amino acid side groups or at their N-terminal or C-terminal end using known techniques. Such derivatives comprise for example aliphatic esters of carboxylic acid groups, amides of carboxylic acid groups, obtainable by reaction with ammonia or with a primary or secondary amine; N-acyl derivatives of free amino groups, produced by reaction with acyl groups; or O-acyl derivatives of free hydroxy groups, produced by reaction with acyl groups.

“Functional equivalents” naturally also comprise polypeptides that can be obtained from other organisms, as well as naturally occurring variants. For example, areas of homologous sequence regions can be established by sequence comparison, and equivalent enzymes can be determined on the basis of the concrete parameters of the invention.

“Functional equivalents” also comprise fragments, preferably individual domains or sequence motifs, of the polypeptides according to the invention, which for example display the desired biological function.

“Functional equivalents” are, moreover, fusion proteins, which have one of the polypeptide sequences stated above or functional equivalents derived there from and at least one further, functionally different, heterologous sequence in functional N-terminal or C-terminal association (i.e. without substantial mutual functional impairment of the fusion protein parts). Non-limiting examples of these heterologous sequences are e.g. signal peptides, histidine anchors or enzymes.

“Functional equivalents” that are also included according to the invention are homologues of the concretely disclosed proteins. These possess percent identity values as stated above. Said values refer to the identity with the concretely disclosed amino acid sequences, and may be calculated according to the algorithm of Pearson and Lipman, Proc. Natl. Acad, Sci. (USA) 85(8), 1988, 2444-2448.

The % identity values may also be calculated from BLAST alignments, algorithm blastp (protein-protein BLAST) or by applying the Clustal setting as given below.

A percentage identity of a homologous polypeptide according to the invention means in particular the percentage identity of the amino acid residues relative to the total length of one of the amino acid sequences concretely described herein.

In the case of a possible protein glycosylation, “functional equivalents” according to the invention comprise proteins of the type designated above in deglycosylated or glycosylated form as well as modified forms that can be obtained by altering the glycosylation pattern.

Such functional equivalents or homologues of the proteins or polypeptides according to the invention can be produced by mutagenesis, e.g. by point mutation, lengthening or shortening of the protein.

Such functional equivalents or homologues of the proteins according to the invention can be identified by screening combinatorial databases of mutants, for example shortening mutants. For example, a variegated database of protein variants can be produced by combinatorial mutagenesis at the nucleic acid level, e.g. by enzymatic ligation of a mixture of synthetic oligonucleotides. There are a great many methods that can be used for the production of databases of potential homologues from a degenerated oligonucleotide sequence. Chemical synthesis of a degenerated gene sequence can be carried out in an automatic DNA synthesizer, and the synthetic gene can then be ligated in a suitable expression vector. The use of a degenerated genome makes it possible to supply all sequences in a mixture, which code for the desired set of potential protein sequences. Methods of synthesis of degenerated oligonucleotides are known to a person skilled in the art (e.g. Narang, S. A. (1983) Tetrahedron 39:3; Itakura et al. (1984) Annu. Rev. Biochem. 53:323; Itakura et al. (1984) Science 198:1056; Ike et al. (1983) Nucleic Acids Res. 11:477).

In the prior art, several techniques are known for the screening of gene products of combinatorial databases, which were produced by point mutations or shortening, and for the screening of cDNA libraries for gene products with a selected property. These techniques can be adapted for the rapid screening of the gene banks that were produced by combinatorial mutagenesis of homologues according to the invention. The techniques most frequently used for the screening of large gene banks, which are based on a high-throughput analysis, comprise cloning of the gene bank in expression vectors that can be replicated, transformation of the suitable cells with the resultant vector database and expression of the combinatorial genes in conditions in which detection of the desired activity facilitates isolation of the vector that codes for the gene whose product was detected. Recursive Ensemble Mutagenesis (REM), a technique that increases the frequency of functional mutants in the databases, can be used in combination with the screening tests, in order to identify homologues (Arkin and Yourvan (1992) PNAS 89:7811-7815; Delgrave et al. (1993) Protein Engineering 6(3):327-331).

### 3.3 Coding Nucleic Acid Sequences

The invention also relates to nucleic acid sequences that code for enzymes as defined herein.

The present invention also relates to nucleic acids with a certain degree of “identity” to the sequences specifically disclosed herein. “Identity” between two nucleic acids means identity of the nucleotides, in each case over the entire length of the nucleic acid.

For example the identity may be calculated by means of the Vector NTI Suite 7.1 program of the company Informax (USA) employing the Clustal Method (Higgins D G, Sharp P M. Fast and sensitive multiple sequence alignments on a microcomputer. Comput Appl. Biosci. 1989 April; 5(2):151-1) with the following settings:

Multiple alignment parameters:

| | |
|---|---|
| Gap opening penalty | 10 |
| Gap extension penalty | 10 |
| Gap separation penalty range | 8 |
| Gap separation penalty | off |
| % identity for alignment delay | 40 |
| Residue specific gaps | off |
| Hydrophilic residue gap | off |
| Transition weighing | 0 |

Pairwise alignment parameter:

| | |
|---|---|
| FAST algorithm | on |
| K-tuple size | 1 |
| Gap penalty | 3 |
| Window size | 5 |
| Number of best diagonals | 5 |

Alternatively the identity may be determined according to Chema, Ramu, Sugawara, Hideaki, Koike, Tadashi, Lopez, Rodrigo, Gibson, Toby J, Higgins, Desmond G, Thompson, Julie D. Multiple sequence alignment with the Clustal series of programs. (2003) Nucleic Acids Res 31 (13):3497-500, the web page: http://www.ebi.ac.uk/Tools/clustalw/index.html# and the following settings

| | |
|---|---|
| DNA Gap Open Penalty | 15.0 |
| DNA Gap Extension Penalty | 6.66 |
| DNA Matrix | Identity |
| Protein Gap Open Penalty | 10.0 |
| Protein Gap Extension Penalty | 0.2 |
| Protein matrix | Gonnet |
| Protein/DNA ENDGAP | −1 |
| Protein/DNA GAPDIST | 4 |

All the nucleic acid sequences mentioned herein (single-stranded and double-stranded DNA and RNA sequences, for example cDNA and mRNA) can be produced in a known way by chemical synthesis from the nucleotide building blocks, e.g. by fragment condensation of individual overlapping, complementary nucleic acid building blocks of the double helix. Chemical synthesis of oligonucleotides can, for example, be performed in a known way, by the phosphoamidite method (Voet, Voet, 2nd edition, Wiley Press, New York, pages 896-897). The accumulation of synthetic oligonucleotides and filling of gaps by means of the Klenow fragment of DNA polymerase and ligation reactions as well as general cloning techniques are described in Sambrook et al. (1989), see below.

The invention also relates to nucleic acid sequences (single-stranded and double-stranded DNA and RNA sequences, e.g. cDNA and mRNA), coding for one of the above polypeptides and their functional equivalents, which can be obtained for example using artificial nucleotide analogs.

The invention relates both to isolated nucleic acid molecules, which code for polypeptides or proteins according to the invention or biologically active segments thereof, and to nucleic acid fragments, which can be used for example as hybridization probes or primers for identifying or amplifying coding nucleic acids according to the invention.

The nucleic acid molecules according to the invention can in addition contain non-translated sequences from the 3′ and/or 5′ end of the coding genetic region.

The invention further relates to the nucleic acid molecules that are complementary to the concretely described nucleotide sequences or a segment thereof.

The nucleotide sequences according to the invention make possible the production of probes and primers that can be used for the identification and/or cloning of homologous sequences in other cellular types and organisms. Such probes or primers generally comprise a nucleotide sequence region which hybridizes under “stringent” conditions (see below) on at least about 12, preferably at least about 25, for example about 40, 50 or 75 successive nucleotides of a sense strand of a nucleic acid sequence according to the invention or of a corresponding antisense strand.

An “isolated” nucleic acid molecule is separated from other nucleic acid molecules that are present in the natural source of the nucleic acid and can moreover be substantially free from other cellular material or culture medium, if it is being produced by recombinant techniques, or can be free from chemical precursors or other chemicals, if it is being synthesized chemically.

A nucleic acid molecule according to the invention can be isolated by means of standard techniques of molecular biology and the sequence information supplied according to the invention. For example, cDNA can be isolated from a suitable cDNA library, using one of the concretely disclosed complete sequences or a segment thereof as hybridization probe and standard hybridization techniques (as described for example in Sambrook, J., Fritsch, E. F. and Maniatis, T. Molecular Cloning: A Laboratory Manual. 2nd edition, Cold Spring Harbor Laboratory, Cold Spring Harbor Laboratory Press, Cold Spring Harbor, N.Y., 1989). In addition, a nucleic acid molecule comprising one of the disclosed sequences or a segment thereof, can be isolated by the polymerase chain reaction, using the oligonucleotide primers that were constructed on the basis of this sequence. The nucleic acid amplified in this way can be cloned in a suitable vector and can be characterized by DNA sequencing. The oligonucleotides according to the invention can also be produced by standard methods of synthesis, e.g. using an automatic DNA synthesizer.

Nucleic acid sequences according to the invention or derivatives thereof, homologues or parts of these sequences, can for example be isolated by usual hybridization techniques or the PCR technique from other bacteria, e.g. via genomic or cDNA libraries. These DNA sequences hybridize in standard conditions with the sequences according to the invention.

“Hybridize” means the ability of a polynucleotide or oligonucleotide to bind to an almost complementary sequence in standard conditions, whereas nonspecific binding does not occur between non-complementary partners in these conditions. For this, the sequences can be 90-100% complementary. The property of complementary sequences of being able to bind specifically to one another is utilized for example in Northern Blotting or Southern Blotting or in primer binding in PCR or RT-PCR.

Short oligonucleotides of the conserved regions are used advantageously for hybridization. However, it is also possible to use longer fragments of the nucleic acids according to the invention or the complete sequences for the hybridization. These standard conditions vary depending on the nucleic acid used (oligonucleotide, longer fragment or complete sequence) or depending on which type of nucleic acid—DNA or RNA—is used for hybridization. For example, the melting temperatures for DNA:DNA hybrids are approx. 10° C. lower than those of DNA:RNA hybrids of the same length.

For example, depending on the particular nucleic acid, standard conditions mean temperatures between 42 and 58° C. in an aqueous buffer solution with a concentration between 0.1 to 5×SSC (1×SSC=0.15 M NaCl, 15 mM sodium citrate, pH 7.2) or additionally in the presence of 50% formamide, for example 42° C. in 5×SSC, 50% formamide. Advantageously, the hybridization conditions for DNA:DNA hybrids are 0.1×SSC and temperatures between about 20° C. to 45° C., preferably between about 30° C. to 45° C. For DNA:RNA hybrids the hybridization conditions are advantageously 0.1×SSC and temperatures between about 30° C. to 55° C., preferably between about 45° C. to 55° C. These stated temperatures for hybridization are examples of calculated melting temperature values for a nucleic acid with a length of approx. 100 nucleotides and a G+C content of 50% in the absence of formamide. The experimental conditions for DNA hybridization are described in relevant genetics textbooks, for example Sambrook et al., 1989, and can be calculated using formulae that are known by a person skilled in the art, for example depending on the length of the nucleic acids, the type of hybrids or the G+C content. A person skilled in the art can obtain further information on hybridization from the following textbooks: Ausubel et al. (eds), 1985, Current Protocols in Molecular Biology, John Wiley & Sons, New York; Hames and Higgins (eds), 1985, Nucleic Acids Hybridization: A Practical Approach, IRL Press at Oxford University Press, Oxford; Brown (ed), 1991, Essential Molecular Biology: A Practical Approach, IRL Press at Oxford University Press, Oxford.

“Hybridization” can in particular be carried out under stringent conditions. Such hybridization conditions are for example described in Sambrook, J., Fritsch, E. F., Maniatis, T., in: Molecular Cloning (A Laboratory Manual), 2nd edition, Cold Spring Harbor Laboratory Press, 1989, pages 9.31-9.57 or in Current Protocols in Molecular Biology, John Wiley & Sons, N.Y. (1989), 6.3.1-6.3.6.

“Stringent” hybridization conditions mean in particular: Incubation at 42° C. overnight in a solution consisting of 50% formamide, 5×SSC (750 mM NaCl, 75 mM trisodium citrate), 50 mM sodium phosphate (pH 7.6), 5×Denhardt Solution, 10% dextran sulfate and 20 g/ml denatured, sheared salmon sperm DNA, followed by washing of the filters with 0.1×SSC at 65° C.

The invention also relates to derivatives of the concretely disclosed or derivable nucleic acid sequences.

Thus, further nucleic acid sequences according to the invention can be derived from the sequences specifically disclosed herein and can differ from it by addition, substitution, insertion or deletion of individual or several nucleotides, and furthermore code for polypeptides with the desired profile of properties.

The invention also encompasses nucleic acid sequences that comprise so-called silent mutations or have been altered, in comparison with a concretely stated sequence, according to the codon usage of a special original or host organism, as well as naturally occurring variants, e.g. splicing variants or allelic variants, thereof.

It also relates to sequences that can be obtained by conservative nucleotide substitutions (i.e. the amino acid in question is replaced by an amino acid of the same charge, size, polarity and/or solubility).

The invention also relates to the molecules derived from the concretely disclosed nucleic acids by sequence polymorphisms. These genetic polymorphisms can exist between individuals within a population owing to natural variation. These natural variations usually produce a variance of 1 to 5% in the nucleotide sequence of a gene.

Derivatives of nucleic acid sequences according to the invention mean for example allelic variants, having at least 60% homology at the level of the derived amino acid, preferably at least 80% homology, quite especially preferably at least 90% homology over the entire sequence range (regarding homology at the amino acid level, reference should be made to the details given above for the polypeptides). Advantageously, the homologies can be higher over partial regions of the sequences.

Furthermore, derivatives are also to be understood to be homologues of the nucleic acid sequences according to the invention, for example animal, plant, fungal or bacterial homologues, shortened sequences, single-stranded DNA or RNA of the coding and noncoding DNA sequence. For example, homologues have, at the DNA level, a homology of at least 40%, preferably of at least 60%, especially preferably of at least 70%, quite especially preferably of at least 80% over the entire DNA region given in a sequence specifically disclosed herein.

Moreover, derivatives are to be understood to be, for example, fusions with promoters. The promoters that are added to the stated nucleotide sequences can be modified by at least one nucleotide exchange, at least one insertion, inversion and/or deletion, though without impairing the functionality or efficacy of the promoters. Moreover, the efficacy of the promoters can be increased by altering their sequence or can be exchanged completely with more effective promoters even of organisms of a different genus.

### 3.4 Constructs According to the Invention

The invention also relates to expression constructs, containing, under the genetic control of regulatory nucleic acid sequences, a nucleic acid sequence coding for a polypeptide or fusion protein according to the invention; as well as vectors comprising at least one of these expression constructs.

“Expression unit” means, according to the invention, a nucleic acid with expression activity, which comprises a promoter as defined herein and, after functional association with a nucleic acid that is to be expressed or a gene, regulates the expression, i.e. the transcription and the translation of this nucleic acid or of this gene. In this context, therefore, it is also called a “regulatory nucleic acid sequence”. In addition to the promoter, other regulatory elements may be present, e.g. enhancers.

“Expression cassette” or “expression construct” means, according to the invention, an expression unit, which is functionally associated with the nucleic acid that is to be expressed or the gene that is to be expressed. In contrast to an expression unit, an expression cassette thus comprises not only nucleic acid sequences which regulate transcription and translation, but also the nucleic acid sequences which should be expressed as protein as a result of the transcription and translation.

The terms “expression” or “overexpression” describe, in the context of the invention, the production or increase of intracellular activity of one or more enzymes in a microorganism, which are encoded by the corresponding DNA. For this, it is possible for example to insert a gene in an organism, replace an existing gene by another gene, increase the number of copies of the gene or genes, use a strong promoter or use a gene that codes for a corresponding enzyme with a high activity, and optionally these measures can be combined.

Preferably such constructs according to the invention comprise a promoter 5′-upstream from the respective coding sequence, and a terminator sequence 3′-downstream, and optionally further usual regulatory elements, in each case functionally associated with the coding sequence.

A “promoter”, a “nucleic acid with promoter activity” or a “promoter sequence” mean, according to the invention, a nucleic acid which, functionally associated with a nucleic acid that is to be transcribed, regulates the transcription of this nucleic acid.

“Functional” or “operative” association means, in this context, for example the sequential arrangement of one of the nucleic acids with promoter activity and of a nucleic acid sequence that is to be transcribed and optionally further regulatory elements, for example nucleic acid sequences that enable the transcription of nucleic acids, and for example a terminator, in such a way that each of the regulatory elements can fulfill its function in the transcription of the nucleic acid sequence. This does not necessarily require a direct association in the chemical sense. Genetic control sequences, such as enhancer sequences, can also exert their function on the target sequence from more remote positions or even from other DNA molecules. Arrangements are preferred in which the nucleic acid sequence that is to be transcribed is positioned behind (i.e. at the 3′ end) the promoter sequence, so that the two sequences are bound covalently to one another. The distance between the promoter sequence and the nucleic acid sequence that is to be expressed transgenically can be less than 200 by (base pairs), or less than 100 by or less than 50 bp.

Apart from promoters and terminators, examples of other regulatory elements that may be mentioned are targeting sequences, enhancers, polyadenylation signals, selectable markers, amplification signals, replication origins and the like. Suitable regulatory sequences are described for example in Goeddel, Gene Expression Technology: Methods in Enzymology 185, Academic Press, San Diego, Calif. (1990).

Nucleic acid constructs according to the invention comprise in particular sequences selected from those, specifically mentioned herein or derivatives and homologues thereof, as well as the nucleic acid sequences that can be derived from amino acid sequences specifically mentioned herein which are advantageously associated operatively or functionally with one or more regulating signal for controlling, e.g. increasing, gene expression.

In addition to these regulatory sequences, the natural regulation of these sequences can still be present in front of the actual structural genes and optionally can have been altered genetically, so that natural regulation is switched off and the expression of the genes has been increased. The nucleic acid construct can also be of a simpler design, i.e. without any additional regulatory signals being inserted in front of the coding sequence and without removing the natural promoter with its regulation. Instead, the natural regulatory sequence is silenced so that regulation no longer takes place and gene expression is increased.

A preferred nucleic acid construct advantageously also contains one or more of the aforementioned enhancer sequences, functionally associated with the promoter, which permit increased expression of the nucleic acid sequence. Additional advantageous sequences, such as other regulatory elements or terminators, can also be inserted at the 3′ end of the DNA sequences. One or more copies of the nucleic acids according to the invention can be contained in the construct. The construct can also contain other markers, such as antibiotic resistances or auxotrophy-complementing genes, optionally for selection on the construct.

Examples of suitable regulatory sequences are contained in promoters such as cos-, tac-, trp-, tet-, trp-tet-, lpp-, lac-, lpp-lac-, lacI^{q-}, T7-, T5-, T3-, gal-, trc-, ara-, rhaP (rhaP_{BAD})SP6-, lambda-P_{R}- or in the lambda-P_{L} promoter, which find application advantageously in Gram-negative bacteria. Other advantageous regulatory sequences are contained for example in the Gram-positive promoters ace, amy and SPO2, in the yeast or fungal promoters ADC1, MFalpha, AC, P-60, CYC1, GAPDH, TEF, rp28, ADH. Artificial promoters can also be used for regulation.

For expression, the nucleic acid construct is inserted in a host organism advantageously in a vector, for example a plasmid or a phage, which permits optimum expression of the genes in the host. In addition to plasmids and phages, vectors are also to be understood as meaning all other vectors known to a person skilled in the art, e.g. viruses, such as SV40, CMV, baculovirus and adenovirus, transposons, IS elements, phasmids, cosmids, and linear or circular DNA. These vectors can be replicated autonomously in the host organism or can be replicated chromosomally. These vectors represent a further embodiment of the invention.

Suitable plasmids are, for example in E. coli, pLG338, pACYC184, pBR322, pUC18, pUC19, pKC30, pRep4, pHS1, pKK223-3, pDHE19.2, pHS2, pPLc236, pMBL24, pLG200, pUR290, pIN-III¹¹³-B1, λgt11 or pBdCl; in nocardioform actinomycetes pJAM2; in Streptomyces pIJ101, pIJ364, pIJ702 or pIJ361; in bacillus pUB110, pC194 or pBD214; in Corynebacterium pSA77 or pAJ667; in fungi pALS1, pIL2 or pBB116; in yeasts 2° al.phaM, pAG-1, YEp6, YEp13 or pEMBLYe23 or in plants pLGV23, pGHlac⁺, pBIN19, pAK2004 or pDH51. The aforementioned plasmids represent a small selection of the possible plasmids. Other plasmids are well known to a person skilled in the art and will be found for example in the book Cloning Vectors (Eds. Pouwels P. H. et al. Elsevier, Amsterdam-New York-Oxford, 1985, ISBN 0 444 904018).

In a further embodiment of the vector, the vector containing the nucleic acid construct according to the invention or the nucleic acid according to the invention can be inserted advantageously in the form of a linear DNA in the microorganisms and integrated into the genome of the host organism through heterologous or homologous recombination. This linear DNA can comprise a linearized vector such as plasmid or just the nucleic acid construct or the nucleic acid according to the invention.

For optimum expression of heterologous genes in organisms, it is advantageous to alter the nucleic acid sequences in accordance with the specific codon usage employed in the organism. The codon usage can easily be determined on the basis of computer evaluations of other, known genes of the organism in question.

The production of an expression cassette according to the invention is based on fusion of a suitable promoter with a suitable coding nucleotide sequence and a terminator signal or polyadenylation signal. Common recombination and cloning techniques are used for this, as described for example in T. Maniatis, E. F. Fritsch and J. Sambrook, Molecular Cloning: A Laboratory Manual, Cold Spring Harbor Laboratory, Cold Spring Harbor, N.Y. (1989) as well as in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc and Wiley Interscience (1987).

The recombinant nucleic acid construct or gene construct is inserted advantageously in a host-specific vector for expression in a suitable host organism, to permit optimum expression of the genes in the host. Vectors are well known to a person skilled in the art and will be found for example in “Cloning Vectors” (Pouwels P. H. et al., Publ. Elsevier, Amsterdam-New York-Oxford, 1985).

### 3.5 Hosts that can be Used According to the Invention

Depending on the context, the term “microorganism” means the starting microorganism (wild-type) or a genetically modified microorganism according to the invention, or both.

The term “wild-type” means, according to the invention, the corresponding starting microorganism, and need not necessarily correspond to a naturally occurring or ganism.

By means of the vectors according to the invention, recombinant microorganisms can be produced, which have been transformed for example with at least one vector according to the invention and can be used for the fermentative production according to the invention.

Advantageously, the recombinant constructs according to the invention, described above, are inserted in a suitable host system and expressed. Preferably, common cloning and transfection methods that are familiar to a person skilled in the art are used, for example co-precipitation, protoplast fusion, electroporation, retroviral transfection and the like, in order to secure expression of the stated nucleic acids in the respective expression system. Suitable systems are described for example in Current Protocols in Molecular Biology, F. Ausubel et al., Publ. Wiley Interscience, New York 1997, or Sambrook et al. Molecular Cloning: A Laboratory Manual. 2nd edition, Cold Spring Harbor Laboratory, Cold Spring Harbor Laboratory Press, Cold Spring Harbor, N.Y., 1989.

The parent microorganisms are typically those which have the ability to produce lysine, in particular L-lysine, from glucose, saccharose, lactose, fructose, maltose, molasses, starch, cellulose or glycerol, fatty acids, plant oils or ethanol. Preferably they are coryneform bacteria, in particular of the genus Corynebacterium or of the genus Brevibacterium. In particular the species Corynebacterium glutamicum has to be mentioned.

Non-limiting examples of suitable strains of the genus Corynebacterium, and the species Corynebacterium glutamicum (C. glutamicum), are

Corynebacterium glutamicum ATCC 13032,
Corynebacterium acetoglutamicum ATCC 15806,
Corynebacterium acetoacidophilum ATCC 13870,
Corynebacterium thermoaminogenes FERM BP-1539,
Corynebacterium melassecola ATCC 17965
and of the genus Brevibacterium, are
Brevibacterium flavum ATCC 14067
Brevibacterium lactofermentum ATCC 13869
Brevibacterium divaricatum ATCC 14020
or strains derived there from like
Corynebacterium glutamicum KFCC10065
Corynebacterium glutamicum ATCC21608

KFCC designates Korean Federation of Culture Collection, ATCC designates American type strain culture collection, FERM BP designates the collection of National institute of Bioscience and Human-Technology, Agency of Industrial Science and Technology, Japan.

The host organism or host organisms according to the invention preferably contain at least one of the nucleic acid sequences, nucleic acid constructs or vectors described in this invention, which code for an enzyme activity according to the above definition.

### 3.6 Fermentative Production of Dipicolinate

The invention also relates to methods for the fermentative production of dipicolinate.

The recombinant microorganisms as used according to the invention can be cultivated continuously or discontinuously in the batch process or in the fed batch or repeated fed batch process. A review of known methods of cultivation will be found in the textbook by Chmiel (Bioprocesstechnik 1. Einführung in die Bioverfahrenstechnik (Gustav Fischer Verlag, Stuttgart, 1991)) or in the textbook by Storhas (Bioreaktoren and periphere Einrichtungen (Vieweg Verlag, Braunschweig/Wiesbaden, 1994)). The culture medium that is to be used must satisfy the requirements of the particular strains in an appropriate manner. Descriptions of culture media for various microorganisms are given in the handbook “Manual of Methods for General Bacteriology” of the American Society for Bacteriology (Washington D.C., USA, 1981).

These media that can be used according to the invention generally comprise one or more sources of carbon, sources of nitrogen, inorganic salts, vitamins and/or trace elements.

Preferred sources of carbon are sugars, such as mono-, di- or polysaccharides. Very good sources of carbon are for example glucose, fructose, mannose, galactose, ribose, sorbose, ribulose, lactose, maltose, sucrose, raffinose, starch or cellulose. Sugars can also be added to the media via complex compounds, such as molasses, or other by-products from sugar refining. It may also be advantageous to add mixtures of various sources of carbon. Other possible sources of carbon are oils and fats such as soybean oil, sunflower oil, peanut oil and coconut oil, fatty acids such as palmitic acid, stearic acid or linoleic acid, alcohols such as glycerol, methanol or ethanol and organic acids such as acetic acid or lactic acid.

Sources of nitrogen are usually organic or inorganic nitrogen compounds or materials containing these compounds. Examples of sources of nitrogen include ammonia gas or ammonium salts, such as ammonium sulfate, ammonium chloride, ammonium phosphate, ammonium carbonate or ammonium nitrate, nitrates, urea, amino acids or complex sources of nitrogen, such as corn-steep liquor, soybean flour, soybean protein, yeast extract, meat extract and others. The sources of nitrogen can be used separately or as a mixture.

Inorganic salt compounds that may be present in the media comprise the chloride, phosphate or sulfate salts of calcium, magnesium, sodium, cobalt, molybdenum, potassium, manganese, zinc, copper and iron.

Inorganic sulfur-containing compounds, for example sulfates, sulfites, dithionites, tetrathionates, thiosulfates, sulfides, but also organic sulfur compounds, such as mercaptans and thiols, can be used as sources of sulfur.

Phosphoric acid, potassium dihydrogenphosphate or dipotassium hydrogenphosphate or the corresponding sodium-containing salts can be used as sources of phosphorus.

Chelating agents can be added to the medium, in order to keep the metal ions in solution. Especially suitable chelating agents comprise dihydroxyphenols, such as catechol or protocatechuate, or organic acids, such as citric acid.

The fermentation media used according to the invention may also contain other growth factors, such as vitamins or growth promoters, which include for example biotin, riboflavin, thiamine, folic acid, nicotinic acid, pantothenate and pyridoxine. Growth factors and salts often come from complex components of the media, such as yeast extract, molasses, corn-steep liquor and the like. In addition, suitable precursors can be added to the culture medium. The precise composition of the compounds in the medium is strongly dependent on the particular experiment and must be decided individually for each specific case. Information on media optimization can be found in the textbook “Applied Microbiol. Physiology, A Practical Approach” (Publ. P. M. Rhodes, P. F. Stanbury, IRL Press (1997) p. 53-73, ISBN 0 19 963577 3). Growing media can also be obtained from commercial suppliers, such as Standard 1 (Merck) or BHI (Brain heart infusion, DIFCO) etc.

All components of the medium are sterilized, either by heating (20 min at 1.5 bar and 121° C.) or by sterile filtration. The components can be sterilized either together, or if necessary separately. All the components of the medium can be present at the start of growing, or optionally can be added continuously or by batch feed.

The temperature of the culture is normally between 15° C. and 45° C., preferably 25° C. to 40° C. and can be kept constant or can be varied during the experiment. The pH value of the medium should be in the range from 5 to 8.5, preferably around 7.0. The pH value for growing can be controlled during growing by adding basic compounds such as sodium hydroxide, potassium hydroxide, ammonia or ammonia water or acid compounds such as phosphoric acid or sulfuric acid. Antifoaming agents, e.g. fatty acid polyglycol esters, can be used for controlling foaming. To maintain the stability of plasmids, suitable substances with selective action, e.g. antibiotics, can be added to the medium. Oxygen or oxygen-containing gas mixtures, e.g. the ambient air, are fed into the culture in order to maintain aerobic conditions. The temperature of the culture is normally from 20° C. to 45° C. Culture is continued until a maximum of the desired product has formed. This is normally achieved within 10 hours to 160 hours.

The cells can be disrupted optionally by high-frequency ultrasound, by high pressure, e.g. in a French pressure cell, by osmolysis, by the action of detergents, lytic enzymes or organic solvents, by means of homogenizers or by a combination of several of the methods listed.

### 3.7 Dipicolinate Isolation

The methodology of the present invention can further include a step of recovering dipicolinate. The term “recovering” includes extracting, harvesting, isolating or purifying the compound from culture media. Recovering the compound can be performed according to any conventional isolation or purification methodology known in the art including, but not limited to, treatment with a conventional resin (e.g., anion or cation exchange resin, non-ionic adsorption resin, etc.), treatment with a conventional adsorbent (e.g., activated charcoal, silicic acid, silica gel, cellulose, alumina, etc.), alteration of pH, solvent extraction (e.g., with a conventional solvent such as an alcohol, ethyl acetate, hexane and the like), distillation, dialysis, filtration, concentration, crystallization, recrystallization, pH adjustment, lyophilization and the like. For example dipicolinate can be recovered from culture media by first removing the microorganisms. The remaining broth is then passed through or over a cation exchange resin to remove unwanted cations and then through or over an anion exchange resin to remove unwanted inorganic anions and organic acids.

### 3.8 Polyester and Polyamine Polymers

In another aspect, the present invention provides a process for the production of polymers, such as polyesters or polyamides (e.g. Nylon®) comprising a step as mentioned above for the production of dipicolinate. The dipicolinate is reacted in a known manner with a suitable co-monomer, as for example di-, tri- or polyamines get polyamides or di-, tri- or polyols to obtain polyesters. For example, the dipicolinate is reacted with polyamine or polyol containing 4 to 10 carbons.

As non-limiting examples of suitable co-monomers for performing the above polymerization reactions there may be mentioned:

polyols such as ethylene glycol, propylene glycol, glycerol, polyglycerols having 2 to 8 glycerol units, erythritol, pentaerythritol, and sorbitol.

polyamines, such as diamines, triamines and tetramines, like ethylene diamine, propylene diamine, butylene diamine, neopentyl diamine, hexamethylene diamine, octamethylene diamine, diethylene triamine, triethylene tetramine, tetraethylene pentamine, dipropylene triamine, tripropylene tetramine, dihexamethylene triamine, amino-propylethylenediamine and bisaminopropylethylenediamine. Suitable polyamines are also polyalkylenepolyamines. The higher polyamines can be present in a mixture with diamines. Useful diamines include for example 1,2-diaminoethane, 1,3-diaminopropane, 1,4-diaminobutane, 1,5-diaminopentane, 1,6-diaminohexane, 1,8-diaminooctane.

The following examples only serve to illustrate the invention. The numerous possible variations that are obvious to a person skilled in the art also fall within the scope of the invention.

### Experimental Part

Unless otherwise stated the following experiments have been performed by applying standard equipment, methods, chemicals, and biochemicals as used in genetic engineering, fermentative production of chemical compounds by cultivation of microorganisms and in the analysis and isolation of products. See also Sambrook et al, and Chmiel et al as cited herein above.

### Example 1

### Cloning of Dipicolinate Synthetase Gene

To enhance the expression of dipicolinate synthetase in C. glutamicum, based on the published B. subtilis sequence (SEQ ID NO:1), a novel spoVF gene of Bacillus subtilis was synthesized, which was adapted to the C. glutamicum codon usage and contained the C. glutamicum sodA promoter and groEL terminator at up- and downstream of the gene, respectively (SEQ ID NO:4). The synthetic spoVF gene showed 75% of similarity on the nucleotide sequence compared with the original Bacillus gene.

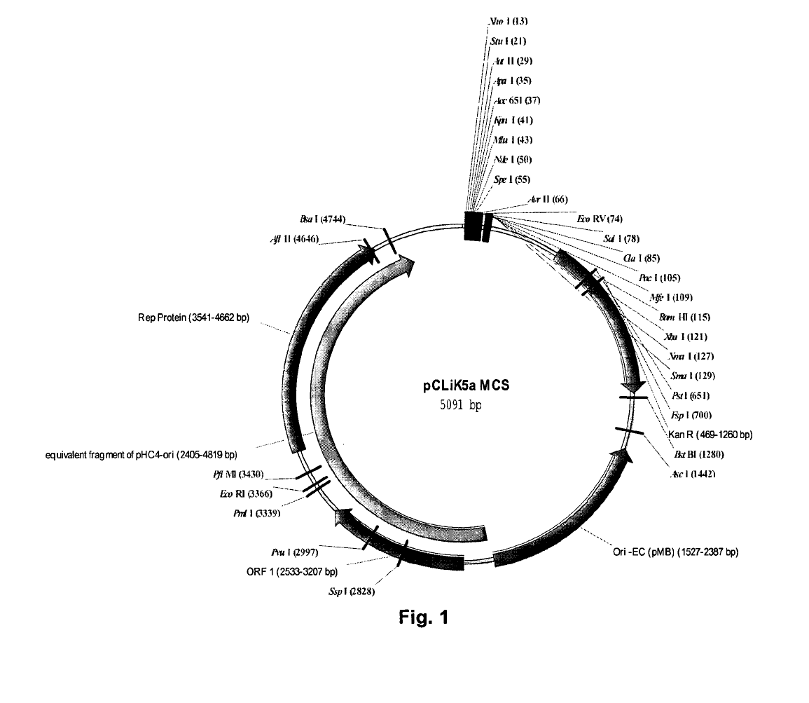

The synthetic spoVF gene was digested with restriction enzyme Spe I, separated on an agarose gel and purified from gel using Qiagen gel extraction kit. This fragment was ligated into the pClik5aMCS vector (SEQ ID NO:7; FIG. 1) previously digested with the same restriction enzyme resulting in pClik5aMCS Psod syn_spoVF.

### Example 2

### Construction of Dipicolinate-Producing Strain

To construct a dipicolinate producing strain, a lysine producer derived from C. glutamicum wild type strain ATCC13032 by incorporation of a point mutation T311I into the aspartokinase gene (NCgl0247), duplication of the diaminopimelate dehydrogenase gene (NCgl2528) and disruption of the phosphoenolpyruvate carboxykinase gene (NCgl2765) was used. Each of said modifications to ATCC 13032 was performed by applying generally known methods of recombinant DNA technology.

Said lysine producer was transformed with the recombinant plasmid pClik5aMCS Psod syn_spoVF of Example 2 by electroporation as described in DE-A-10 046 870.

While the following example is performed with said specifically modified lysine producer strain, other lysine producing strains, well known in the art, may be used as parent strain to be modified by introducing said dipicolinate synthase gene by applying generally known methods of recombinant DNA technology.

Non-limiting suitable further strains to be modified according to the present invention by introducing the dipicolinate synthetase coding sequence are listed above under section 3.5, or are strains described or used in any of the patent applications cross-referenced in the above table under section 3.1, all of which incorporated by reference.

### Example 3

### Dipicolinate Production in Shaking Flask Culture

Shaking flask experiments were performed on the recombinant strains in order to test the dipicolinate production. The strains were pre-cultured on CM plates (10 g/l glucose, 2.5 g/l NaCl, 2 g/l urea, 10 g/l Bacto peptone, 10 g/l yeast extract, 22 g/l agar) for 1 day at 30° C. Cultured cells were harvested in a microtube containing 1.5 ml of 0.9% NaCl and cell density was determined by the absorbance at 610 nm following vortex. For the main culture, suspended cells were inoculated (initial OD of 1.5) into 10 ml of the production medium (40 g/l sucrose, 60 g/1 molasses (calculated with respect to 100° A) sugar content), 10 g/l (NH₄)₂SO₄, 0.6 g/l KH₂PO₄, 0.4 g/l MgSO₄.7H₂O, 2 mg/l FeSO₄.7H₂O, 2 mg/l MnSO₄.H₂O, 0.3 mg/l thiamine.HCl, 1 mg/l biotin) contained in an autoclaved 100 ml of Erlenmeyer flask containing 0.5 g of CaCO₃. Main culture was performed on a rotary shaker (Infors AJ118, Bottmingen, Switzerland) at 30° C. and 220 rpm for 48 hours.

The determination of the dipicolinate concentration was conducted by means of high pressure liquid chromatography according to Agilent on an Agilent 1100 Series LC System. The separation of dipicolinate takes place on an Aqua C18 column (Phenomenex) with 10 mM KH₂PO₄ (pH 2.5) and acetonitrile as an eluent. Dipicolinate was detected at a wavelength of 210 nm by UV detection.

As shown in the following table dipicolinate was accumulated in the broth cultured with the recombinant strain containing spoVF gene.

TABLE
Dipicolinate production in shaking flask culture

| Strains | Dipicolinate (g/l) |
|---|---|
| Lysin producer | 0 |
| +pClik5aMCS | 0 |
| +pClik5aMCS Psod syn_spoVF | 2.1 |

Any document cited herein is incorporated by reference.

1. A method for the fermentative production of dipicolinate, which method comprises the cultivation of a recombinant microorganism, which microorganism is derived from a parent microorganism having the ability to produce lysine via the diaminopimelate (DAP) pathway with L-2,3-dihydrodipicolinate as intermediary product, and wherein the enzyme aspartokinase in the lysine biosynthesis pathway is deregulated, and additionally having the ability to express heterologous dipicolinate synthetase, so that L-2,3-dihydrodipicolinate is converted into dipicolinic acid or a salt thereof.

2. The method of claim 1, wherein said microorganism is a lysine producing bacterium.

3. The method of claim 2, wherein said lysine producing bacterium is a coryneform bacterium.

4. The method of claim 3, wherein the bacterium is a Corynebacterium.

5. The method of claim 4, wherein the bacterium is Corynebacterium glutamicum.

6. The method of claim 1, wherein said heterologous dipicolinate synthetase is of prokaryotic or eukaryotic origin.

7. The method of claim 6, wherein said heterologous dipicolinate synthetase is from a bacterium of the genus Bacillus, in particular from Bacillus subtilis.

8. The method of claim 7, wherein the heterologous dipicolinate synthetase comprises at least one alpha subunit having an amino acid sequence according to SEQ ID NO: 2 or a sequence having at least 80% identity thereto, and at least one beta subunit having an amino acid sequence according to SEQ ID NO: 3 or a sequence having at least 80% identity thereto.

9. The method of claim 1, wherein the enzyme having dipicolinate synthetase activity is encoded by a nucleic acid sequence, which is adapted to the codon usage of said parent microorganism having the ability to produce lysine.

10. The method of claim 1, wherein the enzyme having dipicolinate synthetase activity is encoded by a nucleic acid sequence comprising
a) the spoVF gene sequence according to SEQ ID NO: 1, or
b) a synthetic spoVF gene sequence comprising a coding sequence essentially from residue 193 to residue 1691 according to SEQ ID NO: 4; or
c) a nucleotide sequence encoding a dipicolinate synthetase comprising at least one alpha subunit having the amino acid sequence of SEQ ID NO: 2 or a sequence having at least 80% identity thereto, and at least one beta subunit having the amino acid sequence of SEQ ID NO: 3 or a sequence having at least 80% identity thereto.

11. The method of claim 1, wherein in said recombinant microorganism at least one further gene of the lysine biosynthesis pathway is deregulated.

12. The method of claim 11, wherein said least one deregulated gene selected from aspartatesemialdehyde dehydrogenase, dihydrodipicolinate synthase, dihydrodipicolinate reductase, pyruvate carboxylase, phosphoenolpyruvate carboxylase, glucose-6-phosphate dehydrogenase, transketolase, transaldolase, 6-phosphogluconolactonase, fructose 1,6-biphosphatase, homoserine dehydrogenase, phophoenolpyruvate carboxykinase, succinyl-CoA synthetase, methylmalonyl-CoA mutase, tetrahydrodipicolinate succinylase, succinyl-aminoketopimelate transaminase, succinyl-diaminopimelate desuccinylase, diaminopimelate epimerase, diaminopimelate dehydrogenase, and diaminopimelate decarboxylase.

13. The method of claim 1, wherein the dipicolinate thus produced is isolated from the fermentation broth.

14. A nucleic acid sequence comprising the coding sequence for a dipicolinate synthetase as defined in option b) of claim 10.

15. An expression cassette, comprising at least one nucleic acid sequence as claimed in claim 14, which sequence is operatively linked to at least one regulatory nucleic acid sequence.

16. A recombinant vector, comprising at least one expression cassette as claimed in claim 15.

17. A prokaryotic or eukaryotic host, transformed with at least one vector as claimed in claim 16.

18. The host of claim 17, selected from recombinant coryneform bacteria, especially a recombinant Corynebacterium.

19. The host of claim 18, which is recombinant Corynebacterium glutamicum.

20. A method of preparing a polymer, which method comprises
a) preparing dipicolinate by the method of claim 1;
b) isolating dipicolinate; and
c) polymerizing said dipicolinate with at least one further polyvalent copolymerizable co-monomer

21. The method of claim 20, wherein said copolymerizable co-monomer is selected from polyols and polyamines and mixtures thereof.
